(19) United States
(12) Patent Application Publication
(10) Pub. No.: US 2026/0159793 A1
(43) Pub. Date: Jun. 11, 2026

(54) TREATMENT FACILITY, IN PARTICULAR FOR THE TREATMENT OF ORGANIC WASTE BY FERMENTATION

(71) Applicant: INSTITUT NATIONAL DE RECHERCHE POUR L'AGRICULTURE, L'ALIMENTATION ET L'ENVIRONNEMENT, PARIS (FR)

(72) Inventors: Laurent BLONDEL, SERVON SUR VILAINE (FR); Axelle DEGUEURCE, LA CHAPELLE CHAUSSEE (FR); Pascal PEU, RENNES (FR); Anne TREMIER, GOSNE (FR)

(21) Appl. No.: 19/108,196

(22) PCT Filed: Aug. 1, 2023
(86) PCT No.: PCT/FR2023/051228

§ 371 Date: Feb. 5, 2026

(30) Foreign Application Priority Data
Sep. 2, 2022 (FR) 2208824

(51) Int. Cl. C12M 1/107; C12M 1/00

(52) U.S. Cl. CPC C12M 21/04; C12M 29/00; C12M 29/04

(57) ABSTRACT

A treatment facility (1) for the treatment of fermentable solid products by fermentation has a reactor (2) supported by a frame and a cartridge (11) having an apertured region (110) for storing the product. The reactor (2) is provided with an inlet (3) for products to be treated, an outlet (4) for discharging the treated products, a biogas outlet (5), an opening (8) for inserting an inoculum, and an opening (9) for discharging the leachate. The facility also has a tubular body (6) and a liquid/solid separator (7). The body (6) is closed at both ends by a closing element (100) movable between an open position and a closed position. One of the ends (3) receives the products to be treated and the other of the ends (4) discharges the treated products, each cartridge (11) being slidably movable inside the body (6) and the liquid/solid separator (7) having a filter (12).

The present invention relates to a treatment facility, in particular for the treatment of at least partially fermentable solid products, particularly solid organic waste, by fermentation, particularly anaerobic, in particular with a view to producing biogas. Biogas is given to mean a combustible gas produced by fermentation of organic matter in the absence of oxygen.

The invention relates more particularly to a treatment facility comprising a tubular reactor, a frame for supporting said tubular reactor, and at least one cartridge having a perforated product storage area, said tubular reactor being provided at least with a feed inlet for products to be treated, a first outlet, referred to as the discharge outlet for treated products, a second outlet, referred to as the biogas discharge outlet, a liquid inlet orifice, referred to as the inoculum inlet orifice, and a liquid outlet orifice, referred to as the leachate outlet orifice, said tubular reactor comprising a tubular body and a liquid-solid separator, said tubular body being closed at each of its ends by an airtight closure device, this closure device comprising at least one closure element movably mounted between an open position and a closed position of said end, one of said ends of the body forming the feed inlet for products to be treated and the other forming the discharge outlet for treated products of said reactor, and the or each cartridge being axially slidably mounted inside the body from the feed inlet for products to be treated toward the discharge outlet for treated products.

The transformation of organic matter into biogas, such as methane, by anaerobic fermentation of said organic matter, is a biological process well known to those skilled in the art. This anaerobic fermentation, sometimes also called methanization, is a proven technology for valorizing liquid organic effluent, but improvements remain possible for the valorization of solid organic waste. One of the particular problems in the treatment of solid organic waste lies in the difficulty of placing the microorganisms and the nutrients in contact in an environment in which mechanical agitation is difficult. Methanization facilities based on recycling solid organic waste are however increasingly being developed. In particular, a genuine need exists relating to so-called local methanization facilities. These facilities for the treatment of solid organic waste by anaerobic fermentation are referred to as local in contrast with industrial treatment facilities. These local treatment facilities are intended, in contrast with industrial facilities, for the annual treatment of a smaller quantity of waste, generally of the order of a few hundred tons of waste per year. This results in different requirements for these local treatment facilities, namely in particular: a small footprint, reduced maintenance, simplified use, and a high biogas production rate associated with low water and energy consumption. Until now, the facilities for the treatment of solid organic waste by anaerobic fermentation available on the market, such as the facility described in FR 3 097 230, have not been entirely satisfactory with regard to the requirements set out above.

One aim of the invention is to propose a treatment facility of the aforementioned type the design of which enables a high biogas production rate associated with a reduced quantity of digestate without any detrimental effect on the ease of use of the facility, and low water consumption.

Another aim of the invention is to propose a treatment facility of the aforementioned type the design of which allows easier access to the liquid-solid separator while improving the performance of said separator.

To this end, the invention relates to a treatment facility, in particular for the treatment of at least partially fermentable solid products, particularly solid organic waste, by fermentation, in particular with a view to producing biogas, said facility comprising a tubular reactor, a frame for supporting said tubular reactor, and at least one cartridge having a perforated product storage area, said tubular reactor being provided at least with a feed inlet for products to be treated, a first outlet, referred to as the discharge outlet for treated products, a second outlet, referred to as the biogas discharge outlet, a liquid inlet orifice, referred to as the inoculum inlet orifice, and a liquid outlet orifice, referred to as the leachate outlet orifice, said tubular reactor comprising a tubular body and a liquid-solid separator, said tubular body being closed at each of its ends by an airtight closure device, this closure device comprising at least one closure element movably mounted between an open position and a closed position of said end, one of said ends of the body forming the feed inlet for products to be treated and the other forming the discharge outlet for treated products of said reactor, and the or each cartridge being axially slidably mounted inside the body from the feed inlet for products to be treated toward the discharge outlet for treated products, characterized in that the liquid-solid separator comprises a filter having an active filtration portion configured, in the closed position of the movable closure element of the discharge outlet for treated products of said reactor and in the state in which at least one cartridge is positioned in the reactor, to be interposed between the inoculum inlet orifice and the leachate outlet orifice, and the storage area of the or each cartridge arranged inside the reactor, and in that the inoculum inlet orifice, the leachate outlet orifice, and at least one portion of the filter, are borne by the movable closure element of the discharge outlet for treated products of said reactor.

The active filtration portion of the filter is therefore configured, in the closed position of the movable closure element of the discharge outlet for treated products of said reactor and in the state in which at least one cartridge is positioned in the reactor, to be interposed, that is to form a filtering barrier, between the inoculum inlet orifice and the leachate outlet orifice, and the storage area of the or each cartridge arranged inside the reactor. Thus, any inoculum fed into the reactor must pass through the filtering barrier before it reaches the area for storing products to be treated of each cartridge, and likewise any leachate coming from the product storage area of the cartridge or cartridges arranged inside the reactor must pass through the filtering barrier before it can be discharged from the reactor. As this filtering barrier is active for the filtration of the inoculum and the leachate, which circulate in two opposite directions, this filtering barrier can self-clean under the action of this two-way circulation. The formation of a filter cake is thus slowed. In other words, the filter of the liquid-solid separator of the reactor has an active filtration portion with a first surface configured, in the closed position of the closure elements and in the state in which at least one cartridge is inserted into the reactor, to face toward the product storage area of the or each cartridge positioned inside the reactor and a second opposite surface, and the inoculum inlet orifice and the leachate outlet orifice are positioned on the side of the second surface of said filter. The design of the liquid-solid separator makes it possible, on each immersion operation. to clean the filter during the feeding of the inoculum. The incorporation of at least one portion of the filter of the liquid-solid separator into the movable closure element of the discharge outlet for treated products of the reactor allows easier access to the filter for cleaning or replacement. The design of the facility makes it possible for the facility to operate with reduced manual intervention.

According to one embodiment of the invention, the inoculum inlet orifice and the leachate outlet orifice are common and formed by a single orifice.

This results in the simplification of the facility without any detrimental effect on the operation thereof. This arrangement makes it possible to have a circuit for feeding the inoculum into the reactor that is common with the circuit for discharging the leachate from the reactor so that the circuit is subject to two directions of circulation, which also makes it possible to limit the clogging of the circuit.

According to one embodiment of the invention, the tubular body of the reactor is a cylindrical body of revolution and, in the closed position of the movable closure element of the discharge outlet for treated products of the reactor, the central longitudinal axis of the cylindrical tubular body of the reactor passes through the inoculum inlet orifice and the leachate outlet orifice. In other words, in the closed position of the movable closure element of the discharge outlet for treated products of the reactor, the inlet orifice, the outlet orifice, and the body of the reactor are coaxial.

According to one embodiment of the invention, the facility comprises a liquid storage tank arranged outside the reactor, and a fluid circulation circuit for connecting said tank to the inoculum inlet orifice and the leachate outlet orifice. again, when the orifices are common, this results in the simplification of the facility and the slowing of the clogging of the circuit.

According to one embodiment of the invention, the facility comprises a reversible pump arranged on said fluid circulation circuit, and a unit for controlling said pump. Treatment can thus take place simply by filling the reactor with inoculum until the cartridges contained in the reactor are fully immersed, holding the immersion for a predetermined period, and then draining the reactor.

According to one embodiment of the invention, in the state in which the reactor is closed and the cartridge or cartridges is/are inserted into the reactor, the portion of the filter borne by the movable closure element of the discharge outlet for treated products of said reactor is extended by a porous axial duct arranged inside the reactor. This design makes it possible to increase the filtration surface and reach the area for storing products to be treated of the or each cartridge arranged inside the reactor more quickly.

According to one embodiment of the invention, the or at least one of the cartridges is a cylindrical cartridge internally provided with a central duct extending at least from one end of the cylinder to the opposite end.

According to one embodiment, the central duct of the cartridge is a porous duct, said duct forming at least part of the porous axial duct arranged inside the reactor. This configuration makes it possible to replace one portion of the filter each time a cartridge is removed from the reactor and said cartridge is replaced by a new cartridge. This results in increased efficiency of the filtration.

According to one embodiment of the invention, said cylindrical cartridge comprises a cylindrical body delimited by a perforated wall and at least two perforated covers, one for closing one end and the other for closing the opposite end of the cylinder formed by the body, the wall defining the body is spaced apart from the central duct in order to define a space forming the perforated product storage area of the cartridge, and the ends of the central duct have complementary shapes. The complementary shapes of the ends of the central duct make it possible to connect the central ducts of at least two identical or similar cylindrical cartridges, that is with the same shapes of the ends of the central duct, by nesting. Producing the ends of the central duct with complementary shapes makes it possible to easily ensure the continuity of the axial duct from one cartridge to another, the central duct of each cartridge forming a section of the axial duct. This design also makes it possible, when the reactor contains a plurality of cartridges, to obtain the perfect alignment of the cartridges which, in the state in which they are inserted into the reactor, are arranged aligned one after the other inside said reactor body.

According to one embodiment of the invention, the filter has pores the open area of which is predetermined, and each opening of the wall defining the body of the cartridge and each opening of the closure covers has an open area greater than the open area of any one of the pores of the filter.

According to one embodiment of the invention, the portion of the filter borne by the movable closure element of the discharge outlet for treated products of said reactor is formed by two cones the taper of which is inverted relative to each other so that they are connected by the base of the cones, the vertices of the cones being arranged on a line forming the central longitudinal axis of the tubular body of the reactor in the closed position of the movable closure element of the discharge outlet for treated products. This design makes it possible to limit the formation of a filter cake.

According to one embodiment of the invention, one of the cones forming at least part of the active filtration portion of the filter is delimited by a porous surface, the other cone is delimited by a solid surface, and the inoculum inlet orifice and leachate outlet orifice are arranged at the vertex of the cone having a solid surface.

According to one embodiment of the invention, the facility comprises at least one removable part configured, in the state in which it is positioned inside the reactor body and in the closed position of the movable closure element of the discharge outlet for treated products, to form a spacer between the portion of the filter borne by the movable closure element of the discharge outlet for treated products of said reactor and the cartridge or cartridges arranged inside the reactor. The presence of this spacer facilitates the relative positioning of the active filtration portion and the cartridge nearest to the active filtration portion.

The invention further relates to a treatment method, in particular for the treatment of at least partially fermentable solid products, particularly solid organic waste, by fermentation, in particular with a view to producing biogas, using a facility O the aforementioned type, characterized in that the method comprises, in the state in which one or more cartridges are positioned inside the reactor and in the closed position of the closure elements of the reactor, a step of feeding the reactor with inoculum through the inoculum inlet orifice of said reactor until the cartridge or cartridges is/are immersed in the inoculum, a step of holding the inoculum inside the reactor during a predetermined period, and a step of draining the reactor of leachate through the leachate outlet orifice of said reactor.

### BRIEF DESCRIPTION OF THE DRAWINGS

The invention will be clearly understood on reading the following description of embodiments, with reference to the appended drawings, in which:

FIG. 1 shows a perspective view of a facility according to the invention,

FIG. 2 shows a partial cross-sectional view of a facility according to the invention in the closed position of the closure elements,

FIG. 3 shows a partial perspective view taken from one end of the reactor,

FIG. 4 shows a partial cross-sectional view of a facility according to the invention during the loading of a cartridge in parallel with the unloading of a cartridge,

FIG. 5 shows a partial perspective view of the facility taken from the side of the discharge outlet for treated products of the reactor,

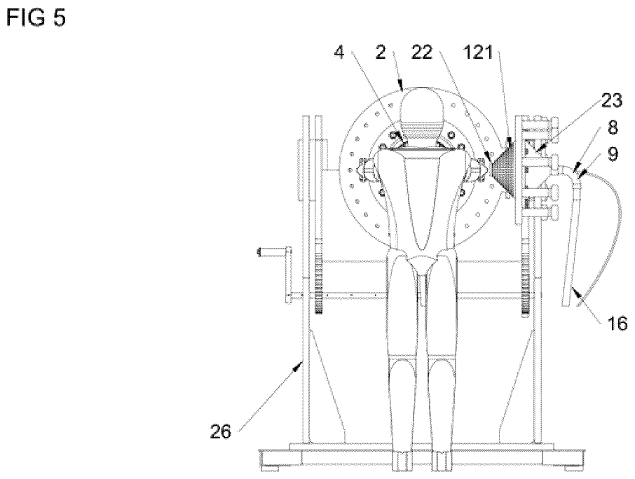

FIG. 6 shows a partial cross-sectional view of the reactor of a facility according to the invention in the absence of cartridges inside the reactor,

FIG. 7 shows a partial cross-sectional view of the liquid-solid separator associated with a spacer and a cartridge,

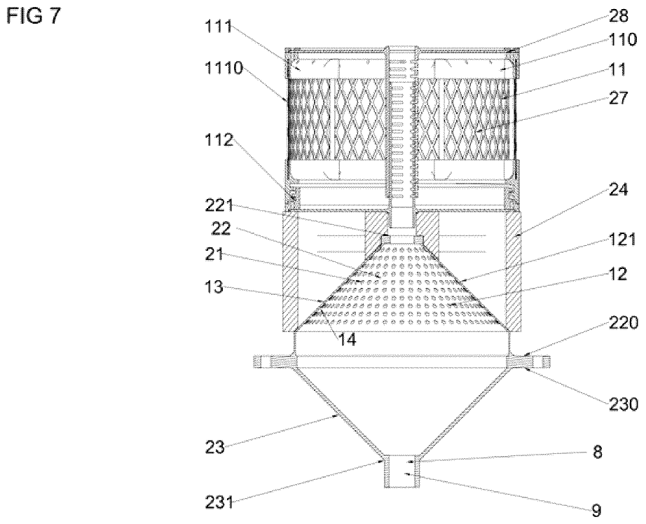

FIG. 8 shows a partial perspective view of the liquid-solid separator associated with a spacer and a cartridge,

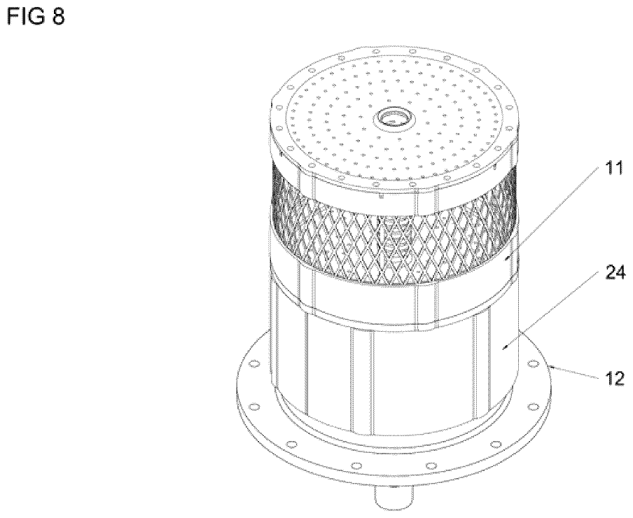

FIG. 9 shows a perspective view of a cartridge in the open state,

FIG. 10 shows a cross-sectional view of a cartridge in the closed state,

FIG. 11 shows a cutaway view of the spacer,

FIG. 12 shows a cross-sectional view of the spacer,

FIG. 13 shows an exploded view of a spacer.

As mentioned above, the treatment facility 1 to which the invention relates is more particularly intended for the treatment, by anaerobic fermentation, of solid organic waste, such as municipal waste, particularly kitchen waste from households, restaurants, and markets comprising waste from preparation waste (peel, pulp) to leftovers, preferably in quantities of 50 to 200 tons per year.

This facility particularly makes it possible, from this fermentation of organic matter in the absence of oxygen, to produce combustible gases essentially comprising methane or carbon dioxide and referred to as biogases.

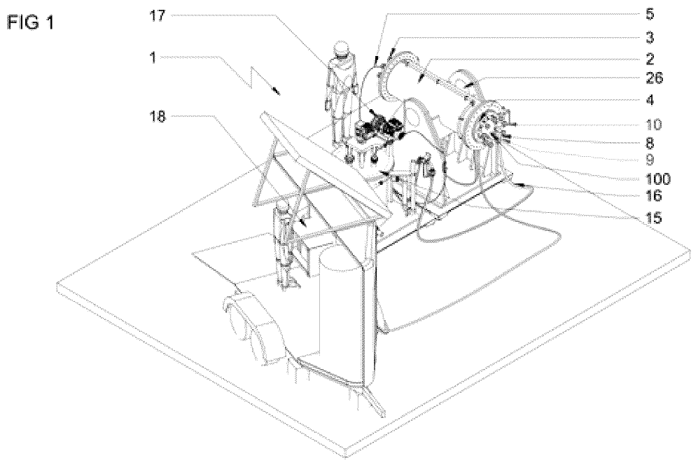

This facility 1, shown in FIG. 1, comprises a tubular reactor 2 and a supporting frame 26 of said tubular reactor 2.

The tubular reactor 2 comprises a tubular body 6 and a liquid-solid separator 7. The tubular body 6 is an elongate body that can have any transverse cross-section. Ideally, this tubular body 6 is a cylindrical body of revolution. The cross-section of said body is circular.

This tubular body 6 is closed at each of its ends by an airtight closure device 10. Each closure device 10 comprises a closure element 100, such as a door or a porthole, movably mounted between an open position and a closed position of said end. In order to move from one position to another, this closure element 100 is articulated on the tubular body 6 of the reactor 2 by an articulated connection, such as a hinge connection.

The reactor 2 has a feed inlet 3 for products to be treated, formed by one of the ends of the body 6, a discharge outlet 4 for treated products formed by the opposite end of the tubular body 6, and a biogas discharge outlet 5.

It should be noted that the feed inlet 3 for products to be treated and the discharge outlet 4 for treated products can be reversed depending on the method of use.

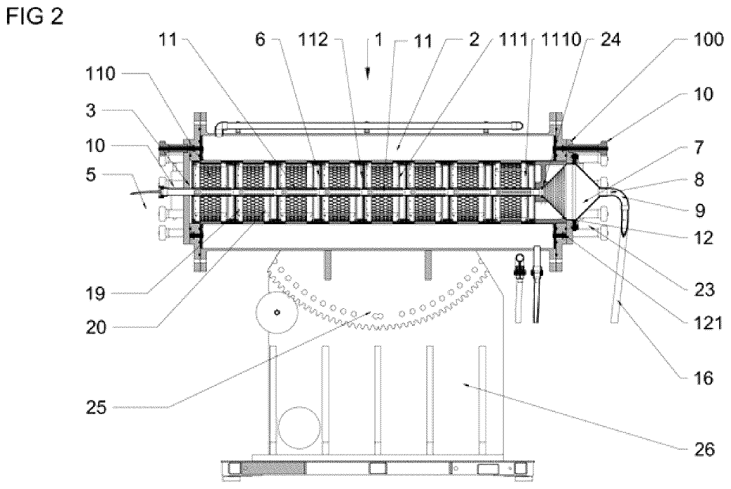

The biogas discharge outlet 5 is, as illustrated in FIG. 2, an axial outlet arranged on the closure element 100 of the feed inlet 3 for products to be treated.

The body 6 of the tubular reactor 2 is, over at least part of its length, delimited by a double wall, and is provided with heating means.

In the example shown in FIG. 1, these heating means are formed by a hot water circuit at least part of which is formed by the space left free between the two walls of the double wall of the body 6 of the tubular reactor.

This heating circuit is fed with hot fluid from a hot fluid tank arranged near the supporting frame 26. This hot fluid tank is provided with members for heating said fluid, such as electrical resistors.

### In the examples shown, the reactor 2 is tiltably

mounted on the supporting frame 26 so that it can be adjusted by means of a pivot connection between the supporting frame 26 and the reactor 2, in order to vary the difference in level between the feed inlet 3 for products to be treated and the discharge outlet 4 for treated products of the reactor 2. This pivot connection has a so-called horizontal pivot axis extending transversely to the longitudinal axis of the body 6 of the reactor and horizontal in the state in which said facility is positioned on a horizontal flat surface.

This ability to tilt the tubular reactor 2 by a pivoting movement about a so-called horizontal pivot axis has many advantages. It makes it possible to reduce the footprint of the reactor, it permits gravitational movement inside said reactor, and it facilitates the reloading of the reactor with products to be treated.

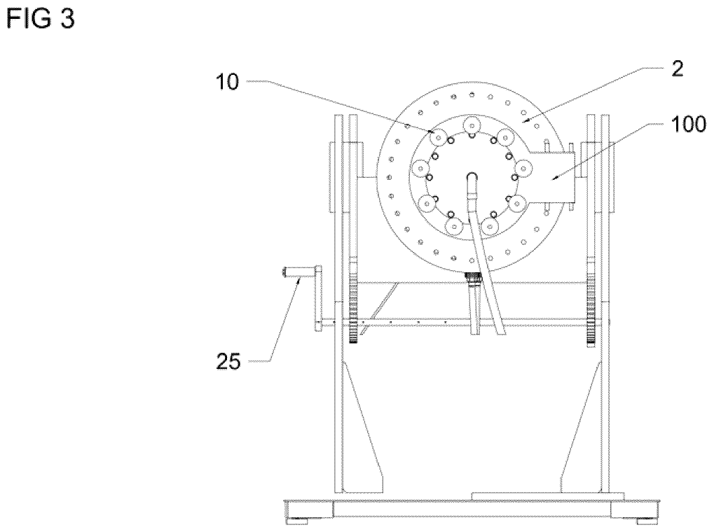

In order to facilitate this pivoting movement of the reactor 2 relative to the supporting frame 26, the facility can comprise a manually or automatically actuated system 25 for driving the pivoting movement of the reactor 2 relative to the supporting frame. This drive system 25 is partially visible in FIGS. 2 and 3.

The pivot through which the pivot axis of the pivot connection between the supporting frame 26 and the reactor 2 passes is rigidly connected to a toothed wheel for rotation therewith. This toothed wheel engages with a pinion driven by a crank and borne by the supporting frame 26. The toothed wheel, the pinion and the crank form the drive system 25.

In order to make it possible to feed the reactor with products to be treated, the treatment facility 1 comprises at least one cartridge 11, preferably a plurality thereof. Each cartridge 11 is inserted into the reactor 2 through the feed inlet 3 for products to be treated and removed from the reactor 2 through the discharge outlet 4 for treated products. Each cartridge 11 is configured, that is shaped and sized, so that it is axially slidable inside the body 6 of the reactor 2 from the feed inlet 3 for products to be treated toward the discharge outlet 4 for treated products.

The cartridges 11 are configured to be arranged one after the other in an aligned state inside the body 6 of the reactor 2. They then form a line of cartridges, so that thrust exerted on the line, at one end of the line of cartridges, causes the movement of the whole line of cartridges.

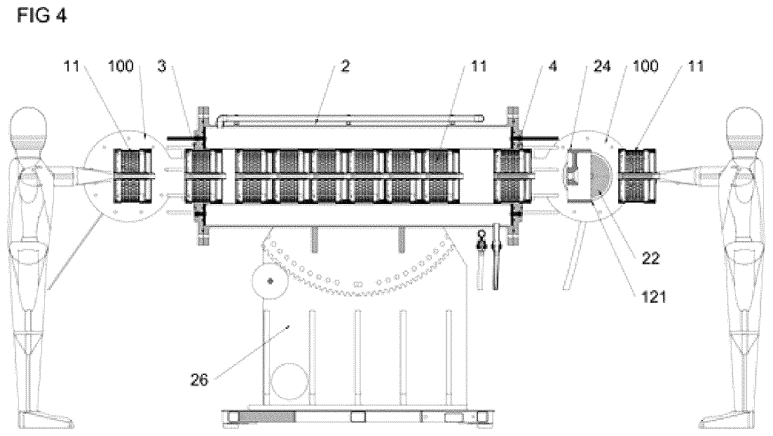

This arrangement makes it possible to remove a cartridge from one end of the body 6 of the reactor 2 in parallel with the entry of a cartridge through the opposite end of the body 6 of the reactor 2, as illustrated in FIG. 4.

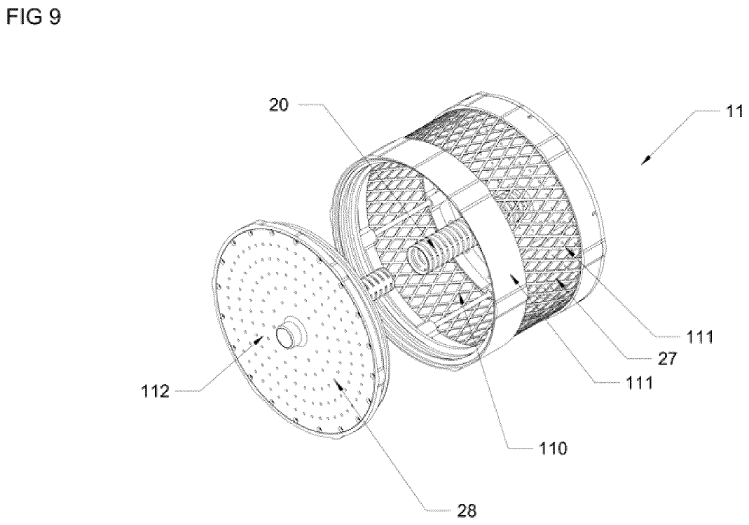

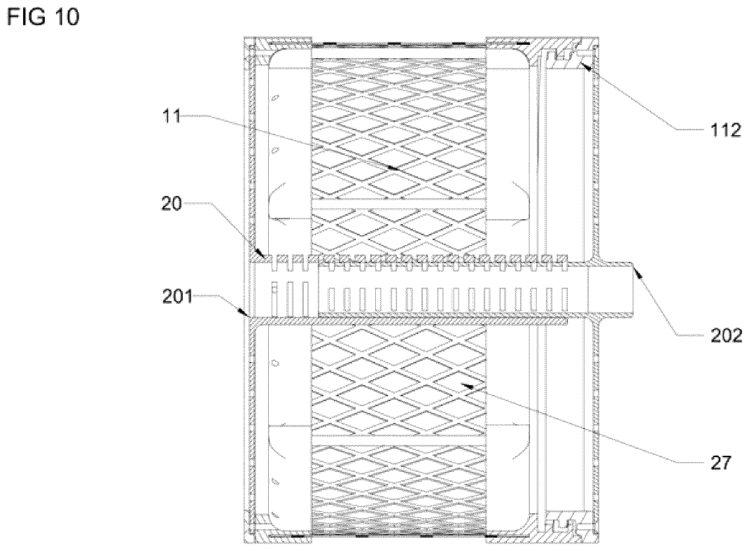

Each cartridge 11, as shown in FIGS. 9 and 10, is a cylindrical cartridge internally provided with a central duct 20 extending at least from one end of the cylinder to the opposite end. This cylindrical cartridge 11 comprises a cylindrical body 111 delimited by a perforated wall 1110 and at least two perforated covers 112, one for closing one end and the other for closing the opposite end of the cylinder formed by the body 111.

One portion of the central duct 20 is integrally formed with one of the perforated covers 112 and another portion of the central duct 20 is integrally formed with the other cover 112.

Said central duct portions are connected to each other by nesting on the closure by screwing of one of the covers 112 to the body of the cartridge.

The continuity of the central duct is thus ensured in the cartridge 11. In the examples shown, the perforated portion of the body of the cartridge is formed by a mesh and each opening 27 of the body corresponds to a cell of the mesh.

The perforated covers 112 in turn have openings 28 formed by perforations.

It will be noted that the ends of the central duct 20, denoted 201 and 202 in the figures, have complementary shapes in order to make it possible to connect the central ducts 20 of at least two identical or similar cylindrical cartridges by nesting.

One end of the central duct 20 protrudes from one of the covers 112 toward the outside of the cartridge in order to facilitate this nesting. In the state in which the cartridges 11 are positioned aligned inside the reactor 2, the central ducts 20 of the cartridges form a continuous duct coaxial with the axis of the body 6 of the reactor 2.

The wall 1110 defining the body of each cartridge 11 is spaced apart from the central duct 20 in order to define a space forming the perforated product storage area of the cartridge. This perforated storage area is denoted 110 in the figures.

After one of the covers of the cartridge has been opened, the products to be treated are therefore inserted into the body of the cartridge in the space between the central duct 20 and the peripheral wall 1110 defining the cylindrical body 111 of the cartridge 11. After the cover has been closed again, the cartridge 11 can be inserted into the reactor 2.

If necessary, in order to facilitate the movement of the products to be treated, and particularly the movement of each cartridge inside the reactor 2, a pusher can be provided, slidably mounted inside the reactor 2.

Due to this pusher, in the state in which it is positioned in the duct of the liquid-solid separator, each cartridge is slidable in said duct of the liquid-solid separator, under the action of thrust exerted by said pusher on said cartridge.

When present, this pusher is a removable pusher that can be removed from or inserted into the reactor 2 through the feed inlet 3 for products to be treated of the reactor 2.

The reactor 2 further comprises a liquid-solid Separator 7 and is provided with a liquid inlet orifice 8, referred to as the inoculum inlet orifice, and a liquid outlet orifice 9, referred to as the leachate outlet orifice 9.

The liquid-solid separator comprises a filter 12 that has an active filtration portion 121 configured, in the closed position of the movable closure element 100 of the discharge outlet 4 for treated products of said reactor 2 and in the state in which at least one cartridge 11 is positioned in the reactor 2, to be interposed between the inoculum inlet orifice 8 and leachate outlet orifice 9, and the storage area 110 of the or each cartridge 11 arranged inside the reactor.

### This active filtration portion 121 therefore forms a

filtering barrier with, on one side of said barrier, the inoculum inlet orifice 8 and leachate outlet orifice 9, and on the other side of the filtering barrier, the storage area 110 of the or each cartridge 11 arranged inside the reactor 2.

The invention is also characterized in that the inoculum inlet orifice 8, the leachate outlet orifice 9, and at least one portion of the filter 12, are borne by the movable closure element 100 of the discharge outlet 4 for treated products of the reactor 2, as shown in FIG. 2.

The active filtration portion 121 of the filter 12 therefore has a first surface 13 facing toward the product storage area 110 of the cartridge or cartridges 11, in the state in which the cartridge or cartridges is/are positioned inside the reactor 2, and an opposite second surface 14.

The inoculum inlet orifice 8 and the leachate outlet orifice 9 are arranged on the side of the second surface 14 of said filter 12.

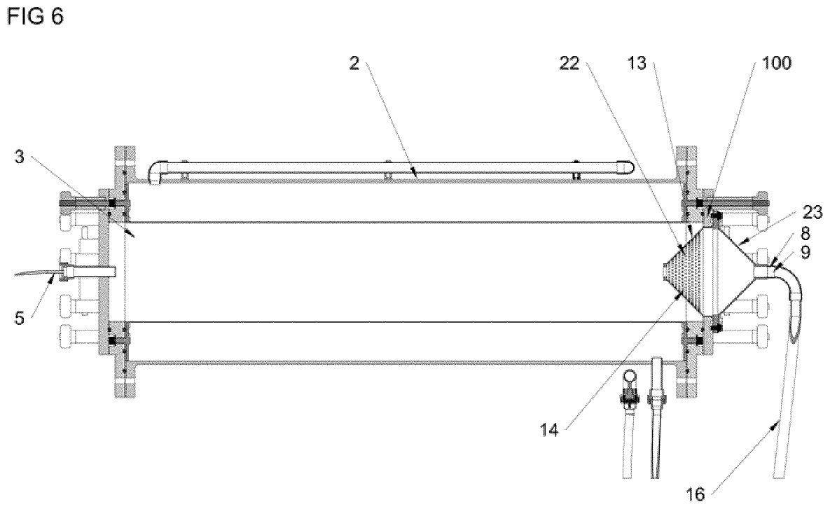

In the example as illustrated in FIG. 6, the portion of the filter 12 borne by the movable closure element 100 of the discharge outlet 4 for treated products of the reactor 2 is formed by two cones 22, 23 the taper of which is inverted relative to each other so that they are connected by the base 220, 230 of the cones.

In the closed position of the movable closure element of the discharge outlet 4 for treated products of the reactor, the vertices 221 and 231 of the cones 22 and 23 are arranged on a line forming the central longitudinal axis of the tubular body 6 of the reactor 2.

One of the cones, denoted 22 in the figures, which forms at least part of the active filtration portion 121 of the filter 12, is delimited by a porous surface. The other cone 23 is delimited by a solid surface.

The inoculum inlet orifice 8 and leachate outlet orifice 9 are arranged at the vertex 231 of the cone 23 having a solid surface. This inoculum inlet orifice 8 and this leachate outlet orifice 9 are common and are formed here by a single orifice.

In the closed position of the movable closure element 100 of the discharge outlet 4 for treated products of the reactor, the central longitudinal axis of the cylindrical tubular body 6 of the reactor 2 passes through the inoculum inlet orifice 8 and the leachate outlet orifice 9.

It will be noted that in the closed position of the closure element 100 of the discharge outlet 4 for treated products, the cone 22 delimited by a porous surface faces toward the inside of the reactor 2 and protrudes inside the body 6 of the reactor 2. In order to make this positioning possible, the articulated connection of the closure element 100 to the body 6 of the reactor is a connection with at least two degrees of freedom in order to permit the pivoting and the translation of the closure element 100 when it moves from one position to another. The cone 23 delimited by a solid surface faces toward the outside of the reactor in the closed position of said reactor.

In order to make it possible to feed inoculum into the inoculum inlet orifice 8 of the reactor and recover the leachate discharged from the reactor through the leachate outlet orifice 9, the facility comprises a liquid storage tank 15 arranged outside the reactor, and a fluid circulation circuit 16 for connecting said tank 15 to the inoculum inlet orifice 8 and the leachate outlet orifice 9.

The facility 1 further comprises a reversible pump 17 arranged on said fluid circulation circuit 16, and a unit 18 for controlling said pump.

The liquid storage tank 15 can be prefilled with inoculum when the facility is started. This inoculum can comprise any liquid digestate originating from a methanization unit situated nearby. Preferably, the digestate used as the inoculum originates from a site that mainly treats kitchen biowaste.

The products to be treated contained in the reactor ferment, in particular on contact with the inoculum, and at least partially liquefy.

This liquid fraction and the injected inoculum form the leachate. This leachate is pumped out of the reactor by the pump 17 that was used to inject the inoculum, and is conveyed to the liquid storage tank 15 before forming the inoculum that will be injected on a new immersion cycle.

Each immersion cycle therefore comprises a step of feeding the reactor with inoculum in a quantity that depends on the desired filling level of the reactor. Generally, this quantity corresponds to the quantity necessary for the storage areas of the cartridges contained in the reactor to be immersed in this inoculum.

The feeding step is followed by a step of holding the inoculum in the reactor during a predetermined period. This second step is followed by a step of draining the reactor to recover the leachate into the liquid storage tank 15. The whole immersion cycle can take place with a single pump 17 and a single storage tank 15.

This storage tank 15 can be provided with a drainage or overflow system in order to control the filling level of said tank. The leachate drawn off from this storage tank 15 can be valorized.

In order to improve the facility, it will be noted that in the example in FIG. 2, that is in the state in which the reactor 2 is closed and the cartridge or cartridges 11 is/are inserted into the reactor 2, the portion of the filter 12 borne by the movable closure element 100 of the discharge outlet 4 for treated products of the reactor 2 is extended by a porous axial duct 19 arranged inside the reactor 2.

This porous axial duct is formed by duct sections, with each section itself being formed by the central duct 20 of a cartridge 11, this central duct 20 being porous.

Preferably, the filter 12 has pores 21 the open area of which is predetermined. Each opening 27 of the wall 1110 defining the body 111 of the cartridge 11 and each opening 28 of the closure covers 112 has an open area greater than the open area of any one of the pores of the filter 12.

By way of example, each opening 27 of the wall 1110 defining the body 111 of the cartridge thus has an area at least equal to 22.5 mm² and each opening 28 of the closure covers 112 has an open area at least equal to 12.6 mm².

The pores of the filter 12 have an open area at most equal to 7.1 mm².

The facility 1 further comprises a removable part 24 configured, in the state in which it is positioned inside the body 6 of the reactor and in the closed position of the movable closure element 100 of the discharge outlet 4 for treated products, to form a spacer between the filter portion 12 borne by the movable closure element 100 of the discharge outlet 4 for treated products of the reactor 2, and the cartridge or cartridges arranged inside the reactor 2.

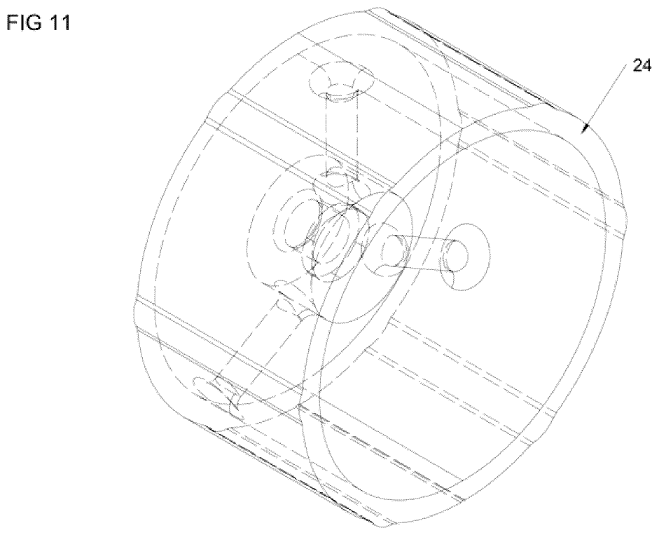

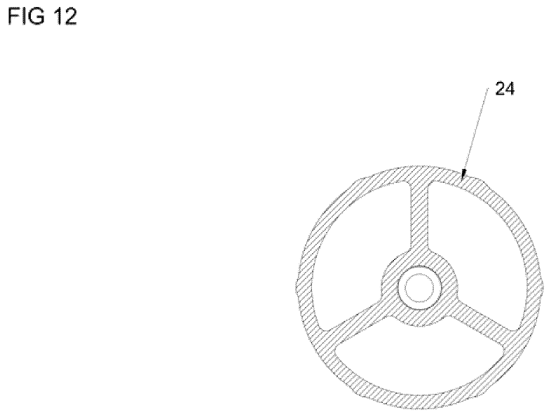

This spacer 24 is shown in detail in FIGS. 11 and 12 in particular. This spacer 24 takes the form of a cylinder section internally provided with a central duct connected to the outer peripheral wall defining the cylinder by radial arms.

In the closed state of the reactor containing at least one cartridge, the central duct of this spacer 24 provides the interface between the central duct of the cartridge nearest to the discharge outlet 4 for treated products of the reactor and the vertex of the cone 22 having a porous surface of the filter 12.

In the examples shown, the central duct of each cartridge is porous, which facilitates the feeding of inoculum to the heart of the products to be treated.

This central duct 20 could be sealed, that is with a solid wall, without departing from the scope of the invention. In this case, the active filtration portion of the filter would be limited to the porous surface of the cone 22.

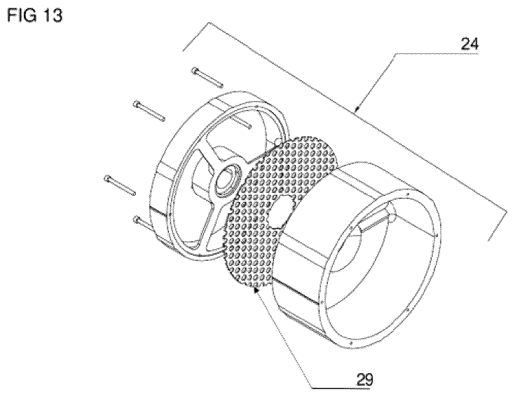

As illustrated in FIG. 13, this spacer 24 can incorporate a centrally pierced filter element 29, here taking the form of a perforated plate.

In practice, such a facility operates as follows.

The cartridges are assumed to have been filled with products to be treated and inserted into the reactor through the feed inlet 3 for products to be treated of the reactor in order to form a line of cartridges inside the reactor, as illustrated in FIG. 2.

Generally, the reactor is arranged vertically with the feed inlet 3 for products to be treated arranged above the discharge outlet 4 for treated products.

An immersion cycle as described above can then be initiated. This cycle comprises a step of feeding the reactor with inoculum by pumping inoculum into the storage tank. The inoculum is introduced into the reactor through the inoculum inlet orifice 8. Part of this inoculum passes through the cone 22 having a perforated surface. Another part passes through the central duct of the cartridges when the duct is perforated in order to reach, in all cases, the product storage area 110 of the cartridges 11.

The fluid coming from the circulation circuit 16 enters the reactor 2 via the inoculum inlet orifice 8, feeds each cartridge via its central duct or its cover, and percolates through the solid products to be treated in each cartridge. It thus passes through the solid products contained in the cartridges until it reaches the cartridge furthest from the inoculum inlet orifice 8.

The distribution of the inoculum in the product storage areas 110 of the cartridges takes place through the pores of the cartridge. Once the desired filling level of the reactor has been reached, the feeding of inoculum is stopped.

When in contact with the solid products to be treated, the inoculum is charged with solubilized organic matter, that is, degraded and liquefied solid organic matter.

Then, after a predetermined period of contact time between the products and the inoculum, the reactor is drained via the leachate outlet orifice 9 after the leachate has passed through the filter 12, and the leachate is collected in the storage tank to form the inoculum of the next immersion cycle.

At the end of an immersion cycle, the decision can be taken to replace one or more cartridges contained in the reactor. The following steps are then simply required:
bringing the reactor to a horizontal position via its pivot drive system,
preparing a trough to be positioned under the closure element of the discharge outlet 4 for treated products in order to recover the leachate stagnating in the tubular reactor when said closure element is opened,
returning this leachate to the storage tank,
opening the closure element of the feed inlet 3 for products to be treated,
removing the spacer,
inserting a cartridge loaded with products to be treated into the reactor through the feed inlet 3 for products to be treated,
recovering, from the discharge outlet 4 for treated products, the cartridge expelled under the effect of thrust exerted on the line of cartridges on the introduction of the new cartridge,
cleaning the cone 22 having a porous surface,
placing the spacer 24 back on the closure element 100 of the discharge outlet 4 for treated products,
closing said closure element 100 of the discharge outlet 4 for treated products again, and
tilting the reactor to the vertical position, the reactor being ready for a new immersion cycle.

Depending on the control methods selected, the reactor can be filled and drained of liquid automatically or manually. In the case of automatic management, the control unit takes the form of an electronic computer system that comprises for example a microprocessor and a working memory. According to one particular aspect, the control unit can take the form of a programmable logic controller. In other words, the functions and steps described can be implemented in the form of a computer program or via hardware components (for example field-programmable gate arrays). In particular, the functions and steps performed by the control unit or its modules can be carried out by sets of instructions or computer modules implemented in a processor or controller, or be carried out by dedicated electronic components or field-programmable gate array (FPGA) or application-specific integrated circuit (ASIC) components. It is also possible to combine computing parts and electronic parts. When it is stated that the unit or means or modules of said unit are configured to perform a given operation, this means that the unit comprises computing instructions and corresponding execution means that make it possible to perform said operation, and/or that the unit comprises corresponding electronic components.

1. A treatment facility, said facility comprising:
a tubular reactor,
a frame for supporting said tubular reactor, and
at least one cartridge having a perforated product storage area,
said tubular reactor being provided at least with a feed inlet for products to be treated,
a first outlet, referred to as the discharge outlet for treated products,
a second outlet, referred to as the biogas discharge outlet,
a liquid inlet orifice, referred to as the inoculum inlet orifice, and
a liquid outlet orifice, referred to as the leachate outlet orifice,
said tubular reactor comprising a tubular body and a liquid-solid separator, said tubular body being closed at each of its ends by an airtight closure device, this closure device comprising at least one closure element movably mounted between an open position and a closed position of said end, one of said ends of the body forming the feed inlet for products to be treated and the other forming the discharge outlet for treated products of said reactor, and the or each cartridge being axially slidably mounted inside the body from the feed inlet for products to be treated toward the discharge outlet for treated products,
wherein that the liquid-solid separator comprises a filter having an active filtration portion configured, in the closed position of the movable closure element of the discharge outlet for treated products of said reactor and in the state in which at least one cartridge is positioned in the reactor, to be interposed between the inoculum inlet orifice and the leachate outlet orifice, and the storage area of the or each cartridge arranged inside the reactor, and in that the inoculum inlet orifice, the leachate outlet orifice, and at least one portion of the filter, are borne by the movable closure element of the discharge outlet for treated products of said reactor.

2. The treatment facility as claimed in claim 1, wherein the inoculum inlet orifice and the leachate outlet orifice are common and formed by a single orifice.

3. The treatment facility as claimed in claim 1, wherein the tubular body of the reactor is a cylindrical body of revolution and in that, in the closed position of the movable closure element of the discharge outlet for treated products of the reactor, the central longitudinal axis of the cylindrical tubular body of the reactor passes through the inoculum inlet orifice and the leachate outlet orifice.

4. The treatment facility as claimed in claim 1, wherein the facility comprises a liquid storage tank arranged outside the reactor and a fluid circulation circuit for connecting said tank to the inoculum inlet orifice and the leachate outlet orifice.

5. The treatment facility as claimed in claim 4, wherein the facility comprises a reversible pump arranged on said fluid circulation circuit, and a unit for controlling said pump.

6. The treatment facility as claimed in claim 1, wherein, in the state in which the reactor is closed and the cartridge or cartridges is/are inserted into the reactor, the portion of the filter borne by the movable closure element of the discharge outlet for treated products of the reactor is extended by a porous axial duct arranged inside the reactor.

7. The treatment facility as claimed in one of claim 1, wherein the cartridge or at least one of the cartridges is a cylindrical cartridge internally provided with a central duct extending at least from one end of the cylinder to the opposite end.

8. The treatment facility as claimed in claim 6,
wherein the cartridge or at least one of the cartridges is a cylindrical cartridge internally provided with a central duct extending at least from one end of the cylinder to the opposite end, and
wherein the central duct of the cartridge is a porous duct, said duct forming at least part of the porous axial duct arranged inside the reactor.

9. The treatment facility as claimed in claim 7, wherein said cylindrical cartridge comprises a cylindrical body delimited by a perforated wall and at least two perforated covers, one for closing one end and the other for closing the opposite end of the cylinder formed by the body, in that the wall defining the body is spaced apart from the central duct in order to define a space forming the perforated product storage area of the cartridge, and in that the ends of the central duct have complementary shapes.

10. The treatment facility as claimed in claim 9, wherein the filter has pores the open area of which is predetermined and in that each opening of the wall defining the body of the cartridge and each opening of the closure covers has an open area greater than the open area of any one of the pores of the filter.

11. The treatment facility as claimed in claim 1, wherein the portion of the filter borne by the movable closure element of the discharge outlet for treated products of said reactor is formed by two cones the taper of which is inverted relative to each other so that they are connected by the base of the cones the vertices of the cones being arranged on a line forming the central longitudinal axis of the tubular body of the reactor in the closed position of the movable closure element of the discharge outlet for treated products.

12. The treatment facility least as claimed in claim 11, wherein one of the cones forming at least part of the active filtration portion of the filter is delimited by a porous surface, in that the other cone is delimited by a solid surface, and in that the inoculum inlet orifice and leachate outlet orifice are arranged at the vertex of the cone having a solid surface.

13. The treatment facility as claimed in claim 1, wherein the facility comprises a removable part configured, in the state in which it is positioned inside the body of the reactor and in the closed position of the movable closure element of the discharge outlet for treated products, to form a spacer between the filter portion borne by the movable closure element of the discharge outlet for treated products of said reactor, and the cartridge or cartridges arranged inside the reactor.

14. The treatment facility as claimed in claim 1, wherein said treatment facility is configured to treat at least partially fermentable solid products, including solid organic waste, by fermentation, for producing biogas.
